(19) United States
(12) Patent Application Publication
(10) Pub. No.: US 2007/0197852 A1
(43) Pub. Date: Aug. 23, 2007

(54) METHOD AND APPARATUS FOR TREATMENT AND DISPOSAL OF WASTE MATERIAL

(76) Inventors: Joseph H. Wilson, Danville, IN (US); Peter B. Weber, Micanopy, FL (US); Gordon I. Kaye, Troy, NY (US); Randall G. McKee, Mooresville, IN (US); William Jones, Avon, IN (US)

Correspondence Address: BINGHAM MCHALE LLP, 2700 MARKET TOWER, 10 WEST MARKET STREET, INDIANAPOLIS, IN, 46204-4900, US

(21) Appl. No.: 11/276,028

(22) Filed: Feb. 10, 2006

(51) Int. Cl. A62D 3/36; B01J 8/08

(52) U.S. Cl. 588/318; 422/198; 422/233

(57) ABSTRACT

A method for the treatment of potentially infectious biological waste material including diseased animal carcasses by comminuting the waste material, mixing the waste with a strong alkaline material and heating is provided. Additionally, a number of apparatuses for practicing the above method wherein biological waste is comminuted, hydrolyzed using a strong alkaline material, and heated by an apparatus mounted to a movable platform are also disclosed.

### CROSS REFERENCE TO RELATED APPLICATIONS

This application is related to U.S. patent application Ser. No. 09/171,447, filed Oct. 20, 1998, titled “Methods for Treatment and Disposal of Regulated Medical Waste,” which claims priority on U.S. provisional patent application Ser. No. 60/178,051, filed Jan. 24, 2001, which are incorporated herein by reference. This application is also related to U.S. patent application Ser. No. 10/263043, filed Oct. 2, 2002, titled “Apparatus and Method for Chemically Reducing Waste Materials,” and U.S. Pat. No. 6,437,211, issued Aug. 20, 2002, which are also incorporated herein by reference.

### FIELD OF THE INVENTION

The present invention relates generally to the field of waste disposal and, more particularly, to a method and apparatus for the treatment and sanitary disposal of infectious waste material such as such as animal byproducts and/or dead or diseased livestock.

### BACKGROUND OF THE INVENTION

Each year millions of animals including, but not limited to, mammals, birds, fish and invertebrates are killed by natural disasters such as floods and hurricanes, man-made disasters such as oil and chemical spills, or by diseases such as hoof and mouth disease, avian influenza, Newcastle's disease and Transmissible Spongiform Encephalopathy (e.g., Bovine TSE or mad cow disease). Events such as these create the need to quickly and safely destroy large numbers of dead and/or diseased animals to prevent the spread of infection to other animals including humans. Additionally, many animal-based agricultural activities such as commercial egg production, meat and poultry processing and commercial fishing and fish farming regularly produce large volumes of animal carcasses and animal byproducts that must be disposed of safely and quickly.

The most common methods currently in use for dealing with large volumes of animal carcasses and animal byproducts are burial, incineration, and rendering. Burial isolates the animal tissue from the environment, but does nothing to destroy actual or potential pathogenic agents. Also, burial leads to decomposition of the animal tissue and an attendant environmental degradation and risk of disease. Incineration is one disposal method that offers the advantage of destroying some or all pathogens present in animal tissues. However, incineration of such large volumes of animal material is generally performed in open trench fires or air curtain burners and under such open-air conditions produces significant air pollution and the possibility of airborne spread of pathogens not destroyed by combustion. Additionally, incineration requires large amounts of fuel to generate the temperatures necessary to destroy animal tissue as well as large areas of land where the open burning can take place. Rendering, a method of cooking animal tissues to reduce their volume before burial or other land-based disposal is also impractical in situations where large numbers of animals must be processed quickly. Rendering requires large, fixed facilities so the animal material must first be transported to the rendering facility before processing.

In some instances, animal tissue to be disposed of is classified as regulated medical waste. Regulated medical waste (hereinafter RMW), pathologic wastes and chemotherapeutic wastes are generally separated from other contaminated components of medical waste and, in most jurisdictions, can only be treated and disposed of by incineration. The developing enforcement regulations derivative from the Clean Air Act and the growing public resistance to the incineration of all RMW, especially the burning of pathogenic and chemotherapeutic wastes (hereinafter Path-Chemo waste), makes the need for development of safe and efficient alternative technologies for treatment of such wastes a primary concern of the healthcare industry. There are currently several acceptable technologies for the treatment and disposal of ordinary RMW, but there is currently no accepted alternative technology to incineration for disposal of Path-Chemo waste. Currently, all Path-Chemo waste must be incinerated.

A need exists for an efficient method of treating large volumes of animal byproducts and/or of dead or diseased animals that is transportable to the site of animal processing, or of a mass animal kill or an agricultural disaster, and operated efficiently. There is also a need for a processing method that would reduce the volume of waste material as well as destroying or otherwise neutralizing any actual or potential pathogens associated with the dead or diseased tissues. There is further a need for an alternative method for the safe, effective, and economical treatment and disposal of large volumes of biologic waste such as that produced by natural disasters, epizootics, or bioterrorism and of pathologic and chemotherapeutic wastes produced in the medical treatment of humans and animals. The present invention addresses these needs.

### SUMMARY OF INVENTION

In one embodiment, a processing apparatus includes a comminution device, a heated reaction chamber, a conveyor for moving waste through the reaction chamber, and a means for injecting an alkaline material into the reaction chamber. In another embodiment, the processing apparatus is mounted to a movable platform such as a truck bed.

One object of the present invention is to provide an improved system, method, and apparatus for disposing of hydrolyzable waste material.

Another object of the present invention is to provide an improved apparatus which is movable and capable of treating and disposing of large numbers of animal carcasses.

Further objects, embodiments, forms, benefits, aspects, features and advantages of the present invention may be obtained from the description, drawings, and claims provided herein.

### BRIEF DESCRIPTION OF THE DRAWINGS

FIG. 1 is a perspective view of one example of a processing apparatus configured in transport mode.

FIG. 2 is a perspective view of another example of a processing apparatus configured in processing mode.

FIG. 3 is a perspective view of yet another example of a processing apparatus.

FIG. 4 is a perspective view of still another example of a processing apparatus.

FIG. 5 is a schematic diagram of a further example of a processing apparatus.

FIG. 6 is a schematic diagram of still another example of a processing apparatus.

### DESCRIPTION OF THE PRESENT INVENTION

For the purposes of promoting an understanding of the principles of the invention and presenting its currently understood best mode of operation, reference will now be made to the embodiments illustrated in the drawings and specific language will be used to describe the same. It will nevertheless be understood that no limitation of the scope of the invention is thereby intended, with such alterations and further modifications in the illustrated device and such further applications of the principles of the invention as illustrated therein being contemplated as would normally occur to one skilled in the art to which the invention relates.

The following discussion relates to a method and apparatus for safely treating and disposing of large volumes of biological material, usually animal carcasses and animal byproducts, produced in animal agriculture and by natural and man-made disasters, agricultural epizootics and panzootics, and bioterrorism. In particular, a method and apparatus for safely treating and disposing of the pathologic and chemotherapeutic wastes (Path-Chemo wastes) produced in human and animal health care either as a separate waste stream or as a component of ordinary regulated medical waste (RMW) is disclosed.

Infectious and potentially infectious and/or noxious materials include animal carcasses or byproducts, human and/or animal pathogenic and/or pathologic wastes, Path-Chemo waste produced in human and veterinary medicine and RMW containing one or more infectious agents including, but not limited to, bacteria, viruses, fungi, Rickettsiae, prokaryotes, infectious proteins, and/or other chemicals and toxins that are capable of causing disease in humans and/or animals. Although the following description refers specifically to the treatment of animal-based wastes, the methods and devices discussed may also be used for the treatment and disposal of other biological materials such as plant materials which may be infected with parasites, fungi, bacteria, viruses, or other pathogens.

The complete destruction or sterilization of microbial life, including highly resistant bacterial endospores, is difficult to prove. Sterilization is instead usually expressed as a probability function in terms of the number of microorganisms surviving a particular treatment process. This function is usually expressed as a logarithmic reduction survival probability in a microbial population. For example, a 6 Log₁₀ reduction is defined as a six decade reduction or a one millionth (0.000001) survival probability for a microbial population (i.e., a 99.9999% reduction). Level III Microbial Inactivation is defined by the State and Territorial Association of Alternative Treatment Technology (hereinafter STAATT) guidelines set out in the STAATT 1 (1994) and STAATT 2 (1998) reports as inactivation of vegetative bacteria, fungi, lipophilic/hydrophilic viruses, parasites and mycobacteria at a 6 Log₁₀ reduction or greater; and inactivation of B. stearothermophilus spores at 4 Log₁₀ reduction or greater.

The method described herein comprises a process of delivering the waste to be treated to a hopper or enclosed entry chamber via dump truck, front end loader, belt conveyor, screw conveyor, cart lifter, or other suitable conveyance means. Also provided is an enclosed connection between the hopper or entry chamber and a chamber containing a hogger, shredder, grinder, or other suitable means to comminute the waste material. The comminuted material next enters a heated reaction chamber containing a variable speed auger or other suitable conveyance device. The heated reaction chamber may comprise a single, jacketed chamber or multiple, jacketed chambers in a stacked or other suitable configuration and connected to one or more comminution devices. The chamber(s) may be jacketed and heated by steam or hot oil, or may be heated by any other suitable heating method. In one example, steam or another suitable heat source also heats the core of the auger or other conveyance device. In another example, the chamber is heated to at least 90° C. to 95° C. In yet another example, the chamber is heated to at least 90° C. to 200° C. In still another example, the chamber is heated to at least 110° C. to 150° C. The comminuted material is moved by the conveyance device through the heated chamber for a sufficient time period to achieve at least Level III inactivation as defined by the STAATT guidelines.

In another example, a basic compound such as dry alkali metal hydroxide, alkali earth metal hydroxide and/or alkali earth metal oxide is added to the waste material. Examples include, but are not limited to, calcium oxide (CaO) (also know as lime or quicklime), magnesium oxide (MgO ) (also know as magnesia), sodium hydroxide (NaOH) (also know as caustic soda), and potassium hydroxide (KOH) (also know as caustic potash). In still a further example, the basic compound is added to the waste material at the beginning of the comminution step such that a solution of alkali in water is formed in the heated chamber as the dry alkaline material dissolves in condensed steam in the chamber. In yet another example, aqueous solutions of alkali metal hydroxide, alkali earth metal hydroxide and/or alkali earth metal oxide are added to the waste material at the beginning of the comminution and/or directly to the heated chamber by an alkali source in fluidic communication with the chamber such as a pipe, nozzle, or other suitable means.

In one example, an alkaline solution having a pH of at least 12 is added. In another example, an alkaline solution having a pH of at least 12-14 is added. One suitable example is a 0.1M to 2.5M aqueous solution of NaOH (i.e., approximately 0.4% to 10% NaOH by weight) giving a solution of pH 13-14. Other solutions having greater or lesser pH values are also contemplated. In still another example, detergents are added to the alkaline solution and waste mixture. Suitable detergents include, but are not limited to, sodium lauryl sulfate (also known as sodium dodecyl sulfate) and deoxycholate (also known as deoxycholic acid, or cholanoic acid). The addition of detergents to the mixture increases the digestion of lipid-soluble wastes and disperses non-saponifiable lipids. In one example, detergents are added to the mixture at a concentration of 0.5% to 5% by weight. In another example, detergents are added to the mixture at a concentration of about 1% by weight.

In still another example, heated steam or liquid is injected into the heated chamber and impinges on the comminuted waste material through the entire length of the heated reaction chamber. In yet another example, the comminuted material is exposed to the heated wall of the chamber and/or heat conducted through the auger or conveyance device by a heat source that is in thermal communication with the conveyance device. Optionally, water is removed from the material as vapor by a negative pressure imposed on a section of the heated chamber by a negative pressure blower or similar air-moving device capable of reducing the pressure in the chamber to below that of the atmosphere. In still another example, the heated chamber is inclined with a low point at the shredder outlet section and a high point at the drying section. In yet another example, the angle of incline of the chamber is adjustable, such as by using hydraulic rams, scissor lifts or other suitable lifting means. In one example, the angle of incline of the chamber is adjustable between zero degrees (i.e., substantially horizontal) and about 30°. Additionally, steam lines and/or hoses and appropriate valves are provided to allow steam cleaning and sterilization of the conveyor, reaction chamber, hopper, connection chamber, shredding chamber and/or shredder.

Optionally, a blower or other suitable air-moving device establishes and maintains a constant airflow through the hopper, connection chamber and/or the upper portion of the shredder. In another example, the air-moving device incorporates a high efficiency particulate air (HEPA) filter or other suitable air purification means. The negative pressure generated by the air-moving device in the hopper, connection chamber and/or the upper portion of the shredder prevents waste particles and potentially infectious materials and agents from being discharged through the material intake portion of the apparatus. Optionally, an interlock mechanism prevents the introduction of raw waste and/or operation of the shredder unless the air-moving device is operating and drawing air through the intake area. In other examples, one or more air-movers or other suitable means for changing the air pressure within the comminution device and/or all or a portion of the heated chamber is also provided.

In another embodiment, the heated chamber is maintained at a near-horizontal position and a baffle is provided at the outflow of the chamber such that a sufficient level of hot alkaline solution is maintained throughout most of the length of the chamber. The comminuted material is bathed by the alkaline solution as the material travels along the chamber from the shredder outlet to the chamber outlet. The hot alkaline solution dissolves animal tissue components and pathologic wastes including, but not limited to, skin, muscle, fat, and internal organs as well as infectious and potentially infectious agents. The alkaline solution causes a significant degree of hydrolysis of the proteins, fats, and nucleic acids of the animal tissues and pathologic wastes and the proteins and nucleic acids of the infectious agents. Additionally, non-protein toxins including, but not limited to, chemotherapeutic agents and other chemicals that may be present in the tissue or RMW, either by exogenous introduction or as synthetic products of infectious agents, are rendered harmless and biodegradable by the action of the hot alkaline solution and/or the hot alkaline solution in combination with the amine groups produced by the hydrolysis of biological tissues. Comminuted, partially digested animal material exiting the chamber is delivered to a storage vessel such as a pit, a lined or unlined trench, a holding tank, or other suitable storage device. In another example, material exiting the jacketed chamber is delivered to a transportable holding device such as a truck, hopper, railcar, barge, manure spreader, or other suitable conveyance. The material is then applied directly to soil, mixed with other amendments and then applied to soil, or transported so that it may be further treated, stored, and/or disposed of as desired.

The comminuted, partially digested material exits the heated chamber in a wet state in one example. Hydrolysis of the material continues in the wet state, even while the material is stored and/or transported, thereby preventing rancidity of the waste material. Any unreacted alkaline solution will ultimately react with carbon dioxide (CO₂) in the atmosphere to produce carbonate salts. For example, if calcium oxide (CaO) is added to the jacketed chamber, excess calcium oxide in the exit material will eventually be converted into calcium carbonate (CaCO₃). Conversion of the unreacted alkaline solution may be increased by passing carbon dioxide through the exit material such as through a bubbler, forced air sparger, or other suitable means. Addition of carbon dioxide to the treated material will also act to reduce the pH of the material.

In another embodiment, the comminuted material is delivered to a heated tank containing a solution of alkali metal hydroxide, alkali earth metal hydroxide and/or alkali earth metal oxide such as those previously described. In one example, the contents of the tank are recirculated using a chopper, grinder pump, or other suitable means. In another embodiment, the tank is a pressure vessel capable of being maintained at an elevated pressure and/or elevated temperature. In one example, the tank is a pressure vessel capable of maintaining a temperature of about 180° C. at a pressure of about 9 bar. In another example, the tank is a pressure vessel capable of maintaining a temperature of about 200° C. at a pressure of about 12 bar. The treated material is then optionally passed through an appropriately porous filter stage before being delivered into the sump of a heated chamber containing a conveyance apparatus such as a variable speed auger. The now sterile waste material is carried though the length of the heated chamber so that heat from the chamber partially or completely dehydrates the sterilized, comminuted material before the material exits the chamber. Water vapor evaporated from the material is removed from the chamber by a negative pressure generated in the chamber by one or more blowers or other suitable air-moving devices. In another example, the material is dried in a drying chamber located after the heated chamber. Optionally, the material is dried using a dryer operationally connected to the chamber such as a basket centrifuge, drum-type vacuum drier, spray dryer, or other suitable drying method. In another example, the angle at which the heated chamber is disposed is adjustable as desired. Typically, the output end of the chamber is elevated above the input end of the chamber so as to control the amount of time material remains in the chamber during the digestion and/or drying step.

Optionally, a device such as a screen or filter for the removal of inorganic materials and/or undigested organic materials is also included. Such materials may include plastics, synthetic fibers, metal, glass, and bone. In one example, such a screen is located at the outlet of the heated chamber.

The reaction rate depends on specific variables such as: the temperature of the waste materials and alkaline solution; the pressure in the heated chamber, sump and/or reaction vessel; the nature of the waste being processed; and the ratio of hydrolyzable material to the volume of alkaline solution. As the reaction rate will vary, the time that the waste must remain in contact with the alkaline solution will also vary. In one example, the amount of time the waste remains in contact with the alkaline solution is adjusted by altering the angle of incline of the heated chamber, adjusting the speed at which the conveyance device is run, or both. In another example, the waste remains immersed within the alkaline solution in the heated chamber until all hydrolyzable matter is fully digested. In yet another example, a slurry of partially digested waste material and alkaline solution exits the heated chamber. The slurry is then stored in a suitable holding device such as a tank car or lined pit as digestion continues. Optionally, the partially digested mixture is further processed using another method, such as by anaerobic fermentation, to produce biogas.

After the waste has been fully digested within the alkaline solution and any solid, undigestible debris removed, the remaining material will comprise a sterile mixture of alkali metal salts of amino acids, sugar acids, nucleotides, small peptides, fatty acids from lipids, phosphates from lipids and nucleic acid breakdown, soluble calcium salts, pigments, sugars, sugar alcohols, hydrocarbons, and inorganic acids derived from the electrolytes normally found within tissue fluids. Infectious agents, including zoonotic agents, prokaryotes, spores, bacteria, viruses, and prions are broken down into low molecular weight residues.

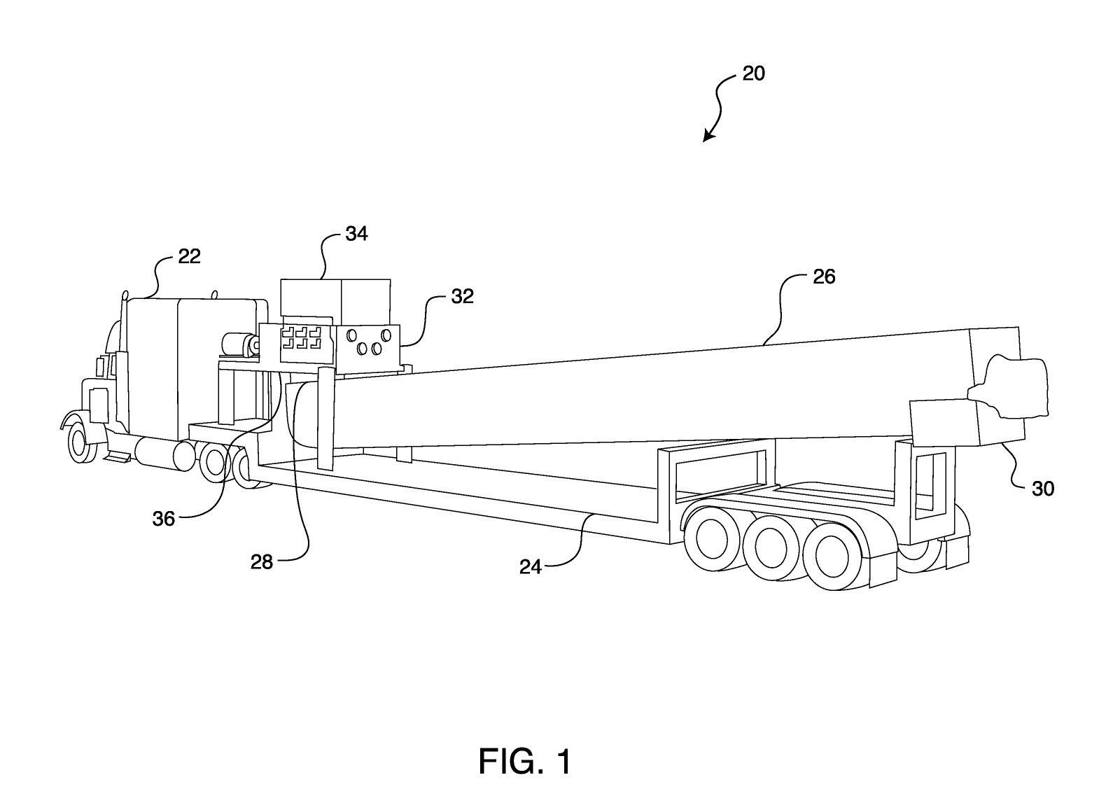

Turning now to the drawings, FIG. 1 shows a perspective view of one embodiment of a processing apparatus 20. This particular embodiment includes a heated chamber 26 having a first opening 28 and a second opening 30 arranged at the opposite end of heated chamber 26 from first opening 28. Processing apparatus 20 further includes a comminution device 32 having a first opening 34 and a second opening 36. In this particular embodiment, comminution device 32 is shown as a shredder. In other embodiments, comminution device 32 is a grinder, hogger, chopper, or other device suitable for processing biological material from large pieces and/or whole animals into smaller pieces. Second opening 36 is configured and arranged such that material processed by comminution device 32 passes through second opening 36 and into first opening 28 of heated chamber 26. In other embodiments, second opening 36 of comminution device 32 and first opening 28 of heated chamber 26 are connected by an enclosure that prevents comminuted materials from exiting apparatus 20.

Continuing with FIG. 1, apparatus 20 is removably mounted to a flatbed trailer 24 of the type that is towed by a tractor-trailer 22. In other embodiments, trailer 24 is movable using agricultural and/or earth moving equipment such as bulldozers or crawlers. In still other embodiments, apparatus 20 is mountable to other, suitable movable platforms such as railcars, barges and the like.

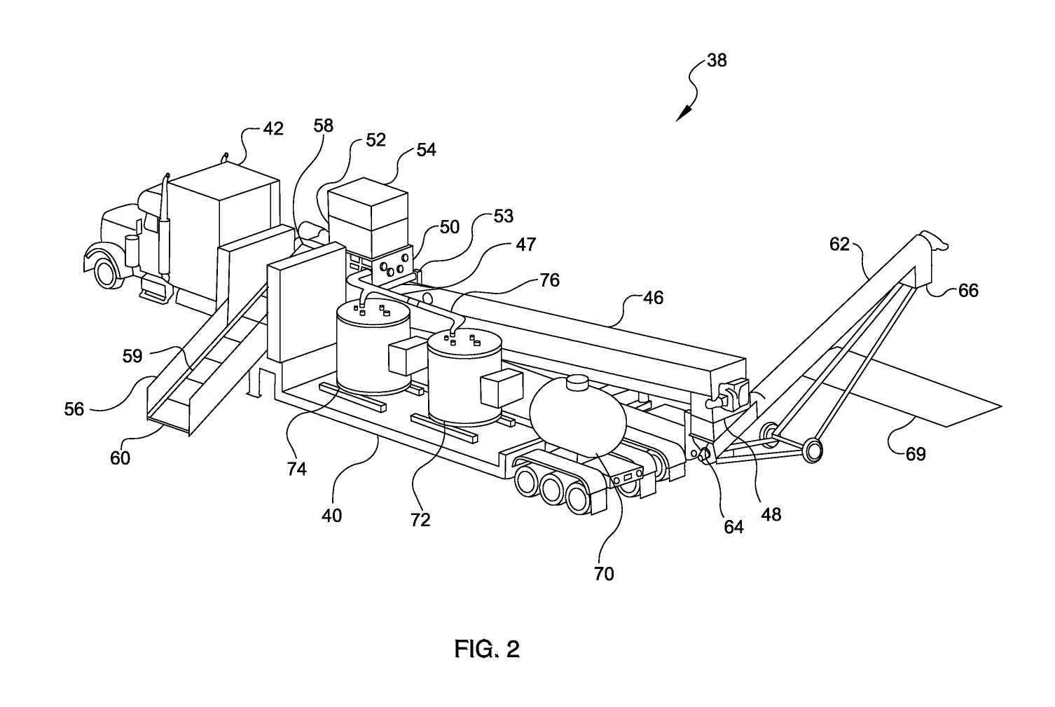

FIG. 2 is a perspective view of a processing apparatus 38 according to another embodiment. In this particular embodiment, processing apparatus 38 includes a heated chamber 46 having a first opening 47 and a second opening 48 arranged at the opposite end of heated chamber 46 from first opening 47. Processing apparatus 38 further includes a comminution device 50 having a first opening 52 and a second opening 53. Second opening 53 is configured and arranged such that material processed by comminution device 50 passes through second opening 53 and into first opening 47 of heated chamber 46.

A hood or enclosure is attached to comminution device 50 so as to cover at least a portion of the device. In this particular embodiment, a hood 54 is shown as attached to comminution device 50 at first opening 52. In other embodiments, a hood or enclosure is attached to comminution device 50 at other locations. In still other embodiments, hood 54 is attached to heated chamber 46. Hood 54 is configured and arranged so as to generate a negative pressure in comminution device 50, heated chamber 46, or in both such that air flows into apparatus 38 through comminution device 50 and into heated chamber 46. The airflow generated by hood 54 prevents particles of biological material created by the actions of comminution device 50 from exiting apparatus 38 without first being processed by the chemical digestion previously described. Hood 54 includes a blower or other air-moving device suitable for generating a negative air pressure in comminution device 50, heated chamber 46, or in both. Optionally, hood 54 further includes an air filtration device such as a HEPA filter, electrostatic precipitator or other suitable air-filtration device.

Processing apparatus 38 further includes a loading apparatus 56. In this particular embodiment, loading apparatus 56 is a belt conveyor having an input 60, an output 58 and a conveyor belt 59. Belt 59 is configured and arranged so as to move materials placed on loading apparatus 56 at input 60 to output 58. Loading apparatus 56 is configured and arranged so that material exiting through output 58 enters comminution device 50 at first opening 52. Loading apparatus 56 is shown as a belt conveyor for illustrative purposes only. Other embodiments contemplate other types of loading apparatuses such as chain-type conveyors, screw conveyors, bucket loaders, and the like. Still other embodiments include a hood or enclosure which covers all or part of loading apparatus 56.

In this particular embodiment, processing apparatus 38 further includes a discharge device 62. Discharge device 62 is shown in this particular example to be an auger, but other types of discharge devices such as pumps, conveyors, and the like are also contemplated. Discharge device 62 includes a first opening 64 and a second opening 66 disposed opposite first opening 64. Discharge device 62 is configured and arranged so that processed material exiting heated chamber 46 through second opening 48 enters discharge device 62 through first opening 64. Optionally, second opening 48 of heated chamber 46 and first opening 64 of discharge device 62 are connected by an enclosure, skirting and/or curtain that prevents processed materials from exiting apparatus 38. Processed material entering discharge device 62 at first opening 64 is conveyed by a screw auger to second opening 66 and discharged. In this particular example, processed material is discharged into an open pit 69. Optionally, open pit 69 is lined with a suitable lining material to prevent seepage. In other examples, processed material is discharged into a hopper, truck bed, rail car, barge or other suitable container as desired. In still other examples, the processing apparatus includes more than one output and may include more than one discharge device.

Continuing with FIG. 2, an auxiliary trailer 40 is also shown. In this particular example, auxiliary trailer 40 is configured to transport utilities for apparatus 38 as well as to transport loading apparatus 56. In other embodiments, loading apparatus 56 is transported separately from auxiliary trailer 40. Optionally, apparatus 38 is configured to utilize on site utilities such as steam, water, electricity, natural gas and the like. As shown, auxiliary trailer 40 is configured to transport a steam generator 74, an alkali storage tank 72 and a fuel storage tank 70. Other configurations and combinations of utilities are also contemplated such as more than one steam generator, alkali storage tank, and/or fuel tank. Optionally, other devices such as diesel engines, hydraulic pumps and electric generators, for example, may also be transported on auxiliary trailer 40.

Fuel storage tank 70 is shown as a propane gas storage tank, although other types of fuel such as diesel are also contemplated. Fuel from storage tank 70 is used to power steam generator 74 and pumps (not shown) which pump alkaline solution from alkali storage tank 72. Optionally, fuel from storage tank 70 is also used to power various pumps, motors and engines utilized by the described process, although utilizing other power sources for the process such as on site electrical power is also contemplated.

Steam from steam generator 74 and alkaline solution from alkali storage tank 72 are delivered to heated chamber 46 through piping array 76. In this particular example, piping array 76 includes pipes for delivering alkali and steam. In other embodiments, separate piping arrays are used. In still other embodiments, a plurality of pipes is used to deliver steam and/or alkaline material to various points in heated chamber 46 and/or a jacket, comminution device 50, or both as desired.

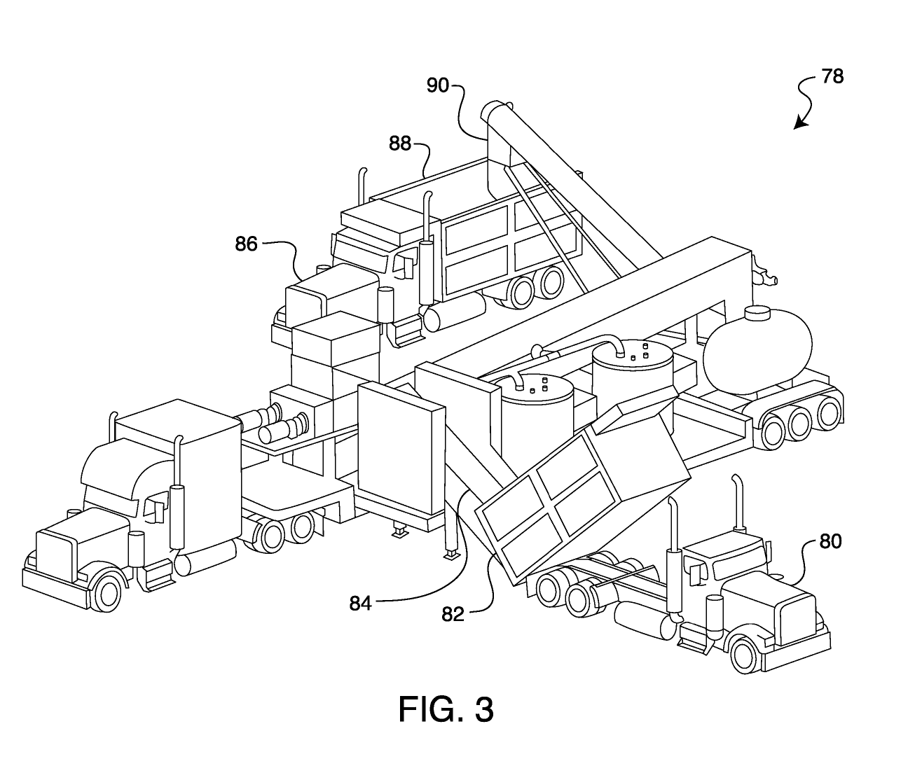

Another embodiment of a processing apparatus 78 is shown in FIG. 3. In this particular embodiment, biological material to be processed is transported to apparatus 78 by a dump truck 80 and unloaded from the truck bed 82 into the apparatus input 84. Apparatus input 84 is shown as a belt conveyor for illustrative purposes only. Processed material exits apparatus 78 through a process output 90 shown in this example as the discharge chute of an auger. Processed material is discharged from output 90 into a truck bed 88 and is hauled away by a dump truck 86.

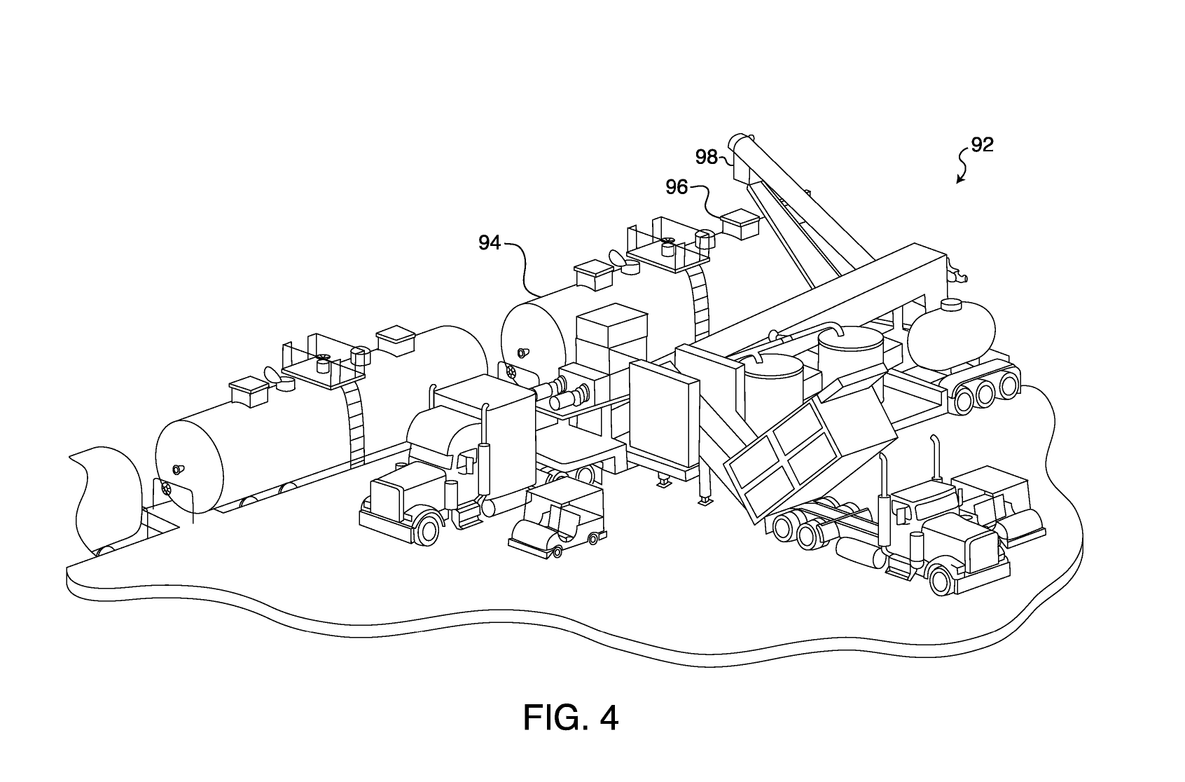

Still another alternative embodiment of a processing apparatus 92 is shown in FIG. 4. In this particular embodiment, processed material exits apparatus 92 through a process output 98 shown in this example as the discharge chute of an auger. Processed material is discharged from output 98 into a tanker rail car 94 through a hatch 96.

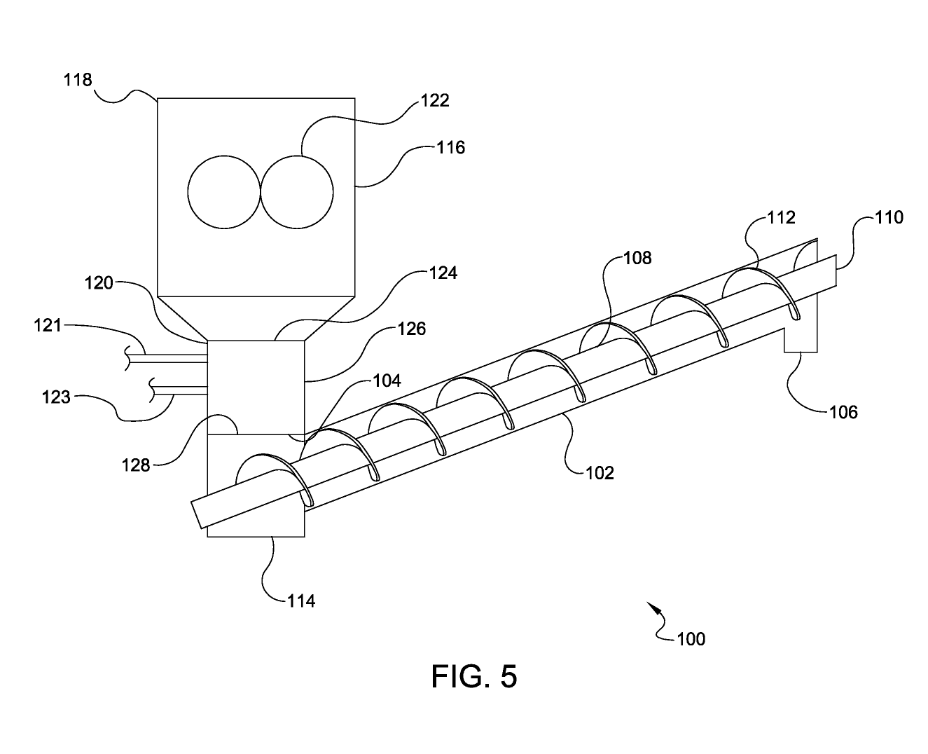

FIG. 5 is a schematic diagram of a processing apparatus 100 according to a further embodiment. This particular embodiment includes a heated chamber 102 having a first opening 104, a sump 114 disposed so that material entering chamber 102 through opening 104 is deposited in sump 114, and a second opening 106 disposed opposite first opening 104. An auger 108, having a shaft 110 and at least one helical blade 112 disposed about shaft 110, is disposed within heated chamber 102. Heat is provided to chamber 102 by a suitable heating means. In one example, heated chamber 102 further includes a jacket that is heated by steam, oil, or another suitable fluid. In another example, heated chamber 102 is heated using electric resistance heaters, induction heaters and the like in thermal communication with the chamber.

Apparatus 100 further includes a comminuting device 116, having a first opening 118 and a second opening 120. In this particular example, comminuting device 116 is a grinder having grinding wheels 122 configured and arranged so that material entering comminuting device 116 through first opening 118 is ground by the movement of grinding wheels 122 before passing through second opening 120. In other examples, comminution device 116 is a chopper, hogger, shredder or other suitable device.

A reaction chamber 126 having a first opening 124 and a second opening 128 is disposed between comminution device 116 and heated chamber 102. Reaction chamber 126 is configured and arranged so that material exiting second opening 120 of comminuting device 116 enters first opening 124 of reaction chamber 126. Reaction chamber 126 is further configured and arranged so that material exiting second opening 128 of reaction chamber 126 enters first opening 104 of heated chamber 102. In this particular embodiment, reaction chamber 126 is a pressure vessel capable of being maintained at an elevated pressure and temperature.

Reaction chamber 126 further includes at least one alkali feed line 123 and at least one steam feed line 121. Alkali feed line 123 is configured and adapted to deliver the desired volume of alkaline material to reaction chamber 126 and is constructed of and/or lined with a material suitable for resisting the caustic nature of the alkaline material. In other examples, alkali feed line 123 is operationally connected to reaction chamber 126, comminution device 116, and/or heated chamber 102 so as to deliver aqueous and/or dry alkaline material as desired. The present example shows a single alkali feed line 123 connected to reaction chamber 126. In other examples, a greater number of alkali feed lines connected to the reaction chamber, heated chamber and/or the comminuting device are used. Steam feed line 121 is sized and adapted to deliver the desired volume of steam to reaction chamber 126. The present example shows a single steam feed line 121 connected to reaction chamber 126. In still other examples, a greater number of steam feed lines connected to the reaction chamber, heated chamber and/or the comminuting device are used. In yet other examples, chamber 126 further includes an air-mover such as a blower configured and arranged to increase the air pressure within the chamber.

Optionally, reaction chamber 126 further includes a means for circulating and/or agitating waste material within the vessel. In one example, the contents of reaction chamber 126 are recirculated using one or more pumps, grinder pumps, or other suitable means. In another example, the contents of reaction chamber 126 are agitated using one or more choppers, mixers, spargers, aerators, or other suitable means.

Treated material exits reaction chamber 126 through opening 128 and enters heated chamber 102 through opening 104 where it is deposited in sump 114. Optionally, treated material is first passed through an appropriately porous filter stage before being delivered into sump 114 of a heated chamber 102. The now sterile waste material is carried though the length of the heated chamber 102 by auger 108 so that heat from the chamber partially or completely dehydrates the sterilized, comminuted material before the material exits chamber 102 through opening 106. Water vapor evaporated from the material is removed from chamber 102 by a negative pressure generated in the chamber by one or more blowers or other suitable air-moving devices (not shown).

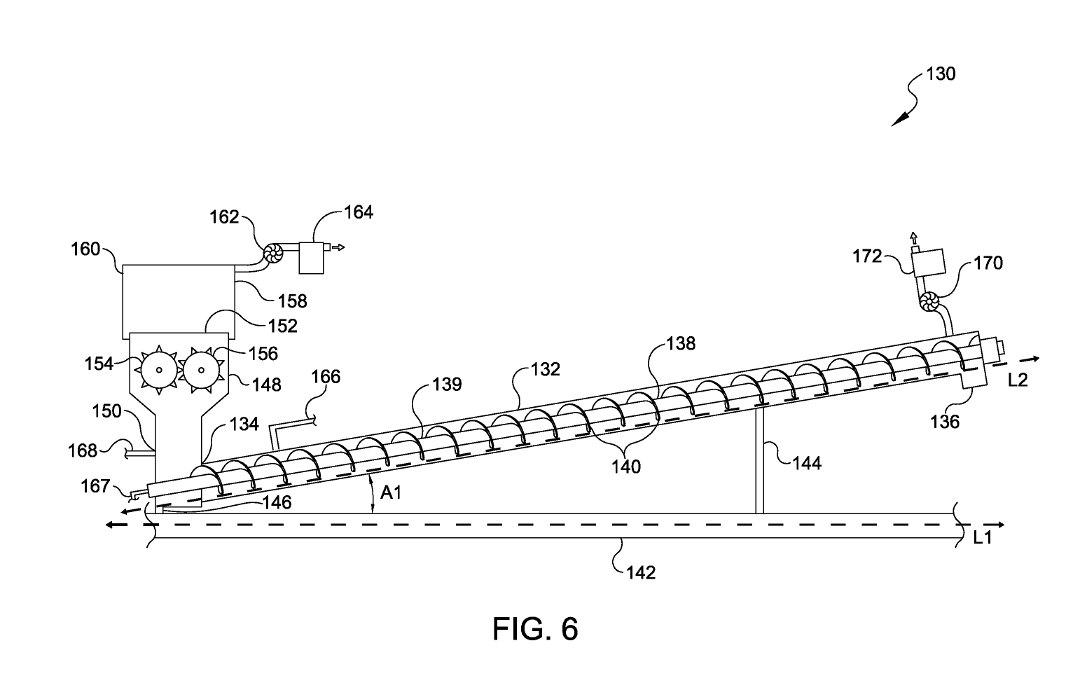

FIG. 6 is a schematic view of a processing apparatus 130 according to another embodiment. This particular embodiment includes a heated chamber 132 having a first opening 134 and a second opening 136 spaced from first opening 134. An auger 138, having a shaft 139 and at least one helical blade 140 disposed about the shaft 139, is disposed within heated chamber 132. A comminuting device 148, having a first opening 152 and a second opening 150, is configured and arranged so that material exiting second opening 150 of comminuting device 148 enters first opening 134 of heated chamber 132. In this particular example, comminuting device 148 is a grinder having two grinding wheels 154, 156 configured and arranged so that material entering comminuting device 148 through first opening 152 is ground by the movement of grinding wheels 154, 156 before passing through second opening 150.

In other embodiments, comminuting device 148 is a shredder, hogger, grinder, or other device capable of grinding, shredding, chopping, or otherwise reducing biological waste to a desired size before the waste enters the heated chamber. In one example, the waste is reduced to pieces no larger than approximately six (6) inches in diameter. In another example, the waste is reduced to pieces no larger than approximately two (2) inches in diameter. The optimal size for waste exiting the comminution device depends on a number of factors including, but not limited to, the nature of the waste being processed, the type of alkali used, whether the alkali is delivered in a dry or aqueous state, the temperature of the heated chamber and the amount of time the waste material will spend in the heated chamber. Generally, comminuting the waste into smaller pieces increases the surface area of the waste relative to the volume, thereby increasing the amount of alkali contacting the waste, increasing the rate of heat transfer into the waste and decreasing processing time.

As shown in FIG. 6, heated chamber 132 is supported by a first support mount 144 and a second support mount 146. First support mount 144 and second support mount 146 are attached to a truck bed 142 using a suitable attachment means such as bolts, welds and the like. Heated chamber 132, first support mount 142 and second support mount 144 are configured and arranged such that a first line L1 substantially parallel to truck bed 142 and a second line L2 substantially parallel to heated chamber 132 intersect to form angle A1. In this particular embodiment, angle A1 is approximately 30°. In other embodiments, angle A1 is equal to or between approximately 0° (i.e., L1 and L2 are substantially parallel) and 90° (i.e., L1 and L2 are substantially perpendicular). In still other embodiments, first support mount 142, second support mount 144 or both are adjustable such that angle A1 can be changed as desired. For example, support mounts are adjustable using scissor lifts, screw or worm gears, hydraulic rams and the like. First support mount 144 and second support mount 146 are shown attached to truck bed 142 for illustrative purposes only. Other examples contemplate mounting heated chamber 132 to other movable bases such as barges and railcars and to non-movable bases such as concrete pads and the like. Also, other embodiments use greater or fewer than two support brackets.

The embodiment shown in FIG. 6 further includes a hood 158 mounted to comminuting device 148 in such a way that any material entering first opening 152 must pass through hood 158. Hood 158 includes an opening 160 allowing biological material to pass through hood 158 and into first opening 152. Optionally, a curtain or screen covers at least a portion of opening 160 to prevent particles of waste from exiting through the opening. A curtain may be made of plastic, rubber or some other suitable, preferably non-porous, material.

In this particular embodiment, a blower 162 is mounted to hood 158. Blower 162 is sized so as to generate an air pressure inside hood 158 slightly below that of atmospheric. Creating a lower than atmospheric pressure in hood 158 draws air through opening 160 and prevents particulate waste matter from exiting apparatus 130 without being processed. Optionally, blower 162 includes an air purification device 164 such as a HEPA filter, electrostatic precipitator, and the like. As shown in FIG. 6, air from hood 158 first passes through blower 162 before passing through air purification device 164. In other embodiments, air passes through air purification device 164 before passing through blower 162. In still other embodiments, a blower and air purification device are attached to the comminuting device and/or the heated chamber.

Continuing with the present example, apparatus 130 further includes at least one alkali feed line 168 and at least one steam feed line 166. Alkali feed line 168 is sized and adapted to deliver the desired volume of alkaline material to apparatus 130 and is constructed of and/or lined with a material suitable for resisting the caustic nature of the alkaline material. As previously described, the alkaline material may be dry or aqueous as desired. The present example shows a single alkali feed line 168 connected to heated chamber 132. In other examples, a greater number of alkali feed lines connected to the heated chamber and/or the comminuting device are used. Steam feed line 166 is sized and adapted to deliver the desired volume of steam to apparatus 130. The steam heats the waste material to speed digestion and processing. In embodiments using dry alkaline material, steam also dissolves the alkali to create a basic solution. The present example shows a single steam feed line 166 connected to heated chamber 132. In other examples, a greater number of steam feed lines connected to the heated chamber and/or the comminuting device are used. In still other examples, a heated fluid other than steam is used. In yet another example, heated chamber 132 further includes a jacket that is heated by steam, oil, or another suitable fluid. Alternatively, heated chamber 132 is heated using electric resistance heaters, induction heaters and the like in thermal communication with the chamber.

The present example further includes a steam feed line 167 mounted to shaft 138 of auger 139. Heating shaft 138 raises the temperature of the waste material being processed thereby speeding digestion. Optionally, one or more sections of blade 140 are perforated or otherwise configured so as to allow steam from feed line 167 to pass through shaft 138 and into heated chamber 132. In other embodiments, a feed line configured to deliver a heated fluid other than steam, such as oil, is mounted to the auger shaft. In still other examples, shaft 138 is in thermal communication with another heat source such as resistance heaters, induction heaters and the like.

Continuing with the apparatus show in FIG. 6, a blower 170 is mounted to heated chamber 132 proximal to opening 136. Blower 170 is sized so as to generate an air pressure inside heated chamber 132 slightly below that of atmospheric. Creating a lower than atmospheric pressure in heated chamber 132 acts to dehydrate the processed waste by drawing water vapor from the slurry before it exits the chamber. The moisture content of the processed waste can be controlled as desired by adjusting the amount of water vapor removed by blower 170. Optionally, blower 170 includes an air purification device 172 such as a HEPA filter, electrostatic precipitator, and the like. In other embodiments, blower 170 further includes a means for collecting the water vapor removed from the slurry such as a condenser. Optionally, the collected water is drawn off for disposal or recycled to the apparatus. As shown in FIG. 6, air from heated chamber 132 first passes through blower 170 before passing through air purification device 172. In other embodiments, air passes through air purification device 172 before passing through blower 170.

While the invention has been illustrated and described in detail in the drawings and foregoing description, the same is to be considered as illustrative and not restrictive in character. It is understood that the embodiments have been shown and described in the foregoing specification in satisfaction of the best mode and enablement requirements. It is understood that one of ordinary skill in the art could readily make a nigh-infinite number of insubstantial changes and modifications to the above-described embodiments and that it would be impractical to attempt to describe all such embodiment variations in the present specification. Accordingly, it is understood that all changes and modifications that come within the spirit of the invention are desired to be protected.

What is claimed is:

1. A method for treating biological waste material, comprising the steps of:
a) comminuting the waste material;
b) providing a highly alkaline material;
c) contacting the waste material with the highly alkaline material;
d) agitating the waste material;
e) heating the waste material; and
f) depositing the waste material in a storage vessel.

2. The method of claim 1, wherein the highly alkaline material has a pH of at least 12.

3. The method of claim 1, further comprising prior to step f) the step of drying the waste material.

4. The method of claim 1, further comprising after step a) and prior to step f) the step of subjecting the waste material to pressure greater than atmospheric pressure.

5. The method of claim 4 wherein the pressure is approximately 1 to 9 bar.

6. The method of claim 4 wherein the waste material is heated to at least 90° C.

7. The method of claim 6 wherein the waste material is heated to between about 110° C. and about 150° C.

8. The method of claim 1 wherein the highly alkaline material is heated prior to step b).

9. A device for the treatment of biological waste material, comprising:
a chamber having a first opening and a second opening spaced from the first opening;
a comminution device configured and arranged to deliver waste material to the first opening of the chamber;
a conveyor configured and arranged to move waste material from the first opening to the second opening of the chamber;
an alkali source in operational communication with the chamber; and
a heat source in thermal communication with the chamber.

10. The device of claim 9, wherein the conveyor comprises an auger.

11. The device of claim 9, further comprising a dryer operationally connected to the chamber.

12. The device of claim 9, wherein the heat source uses steam.

13. The device of claim 9, further comprising an air-moving device configured and arranged to change the air pressure in at least a portion of the chamber.

14. The device of claim 9, further comprising an air-moving device configured and arranged to lower the air pressure in the comminution device below atmospheric pressure.

15. The device of claim 14, further comprising a hood covering at least a portion of the comminution device.

16. The device of claim 9, wherein the chamber and the comminution device are mounted to a movable platform.

17. The device of claim 16, wherein the movable platform is a truck bed.

18. A device for the treatment of biological waste material, comprising:
a comminution device having an input configured and arranged to accept biological waste and an output;
a first chamber having an input configured and arranged to receive waste from the output of the comminution device and an output;
a second chamber having an input configured and arranged to receive waste material from the output of the first chamber and an output;
a conveyor configured and arranged to move waste from the input of the second chamber to the output of the second chamber;
at least one alkali feed line configured and arranged to deliver an alkaline material to the first chamber;
at least one air-mover configured and arranged to increase the pressure in the first chamber above atmospheric pressure; and
at least one heater in thermal communication with the second chamber;
wherein the comminution device, the first chamber and the second chamber are mounted to a movable platform.

19. The device of claim 18, further comprising a hood which encloses at least a portion of the comminution device and an air-mover capable of producing a pressure lower than atmospheric pressure in the comminution device.

20. The device of claim 18, wherein the movable platform is a truck bed.
